(19) United States
(12) Patent Application Publication
(10) Pub. No.: US 2021/0050088 A1
(43) Pub. Date: Feb. 18, 2021

(54) PATIENT-BASED DIETARY PLAN RECOMMENDATION SYSTEM

(71) Applicant: Société des Produits Nestlé S.A., Vevey (CH)

(72) Inventors: Mary Christ, Forham Park, NJ (US); Maureen Huhmann, Branchburg, NJ (US)

(21) Appl. No.: 16/538,115

(22) Filed: Aug. 12, 2019

(51) Int. Cl. G16H 20/60; G16H 50/70; G16H 50/20; G16H 10/20

(52) U.S. Cl. CPC G16H 20/60; G16H 10/20; G16H 50/20; G16H 50/70

(57) ABSTRACT

Methods and systems for generating patient-based dietary plan recommendations are presented. In one embodiment, a method is provided that includes identifying user information for a user. The user information may indicate a symptom affecting the user. The symptom may be used to identify a dietary requirement. The method may then proceed with identifying a recipe requirement based on the dietary requirement and presenting a recipe recommendation to the user based on the recipe requirement.

### BACKGROUND

Patients diagnosed with certain medical conditions (e.g., cancers, digestive conditions) often experience one or more symptoms that make eating typical diets difficult. Additionally, to treat these medical conditions, patients may undergo one or more treatments (e.g., chemotherapy, surgery), which may interfere with their ability to eat certain foods.

### SUMMARY

The present disclosure presents new and innovative methods and systems for personalized dietary plan recommendations for patients. In one embodiment, a method is provided including identifying user information indicating a symptom affecting a user and identifying a dietary requirement based on the symptom. The method may further include identifying a recipe requirement based on the dietary requirement and presenting a recipe recommendation to the user based on the recipe requirement.

In another embodiment, identifying the user information further includes one or both of receiving user information from the user indicating the symptom and identifying previously-received user information indicating the symptom.

In yet another embodiment, the method further includes identifying a plurality of recipes within a recipe selection database that comply with the recipe requirement, selecting at least one selected recipe from among the plurality of recipes, and including the at least one selected recipe in the recipe recommendation.

In a further embodiment, the at least one selected recipe is selected from among the plurality of recipes according to a user preference associated with the user information.

In a still further embodiment, the method includes receiving an initial recipe from a recipe database, extracting an ingredients list and an associated tag from the initial recipe, and generating nutrition information and preparation instructions based on the ingredients list and the associated tag.

In another embodiment, the method further includes combining the nutrition information and the preparation instructions with the ingredients list and the associated tag to form a generated recipe.

In yet another embodiment, the method further includes storing the generated recipe in a recipe selection database.

In a further embodiment, the dietary requirement identifies types of (i) recipes or (ii) food attributes that are associated with alleviating or resolving the symptom.

In a still further embodiment, the recipe requirement identifies one or more excluded ingredients, included ingredients, excluded ingredient types, included ingredient types, and/or nutritional requirements to comply with the dietary requirement.

In another embodiment, the symptom includes at least one condition selected from the group consisting of poor appetite, xerostomia, weight loss, mucositis, nausea, dysphagia, constipation, and diarrhea.

In yet another embodiment, a system is provided comprising a processor; and a memory. The memory may store instructions which, when executed by the processor, cause the processor to implement a recommendation requirements database including at least (i) a dietary requirements table storing a plurality of dietary requirements associated with one or more symptoms and (ii) a recipe requirements table storing a plurality of recipe requirements associated with the dietary requirements. The memory may further store instructions which, when executed by the processor, cause the processor to implement a user recommendation system configured to identify user information indicating a symptom affecting a user and identify, within the dietary requirements table, a dietary requirement based on the symptom. The user recommendation system may be further configured to identify, within the recipe requirements table, a recipe requirement based on the dietary requirement and presenting a recipe recommendation to the user based on the recipe requirement.

In a further embodiment, the user recommendation system is configured, to identify the user information by receiving user information from the user indicating the symptom and identifying previously-received user information indicating the symptom.

In a still further embodiment, the memory stores further instructions which, when executed by the processor, cause the processor to further implement a recipe selection database storing a plurality of recipes associated with the plurality of recipe requirements. The user recommendation system may be further configured to identify a plurality of recipes within the recipe selection database that comply with the recipe requirement, select at least one selected recipe from among the plurality of recipes, and include the at least one selected recipe in the recipe recommendation.

In another embodiment, the at least one selected recipe is selected from among the plurality of recipes according to a user preference associated with the user information.

In yet another embodiment, the memory stores further instructions which, when executed by the processor, cause the processor to further implement a recipe generation system configured to receive an initial recipe from a recipe database, extract an ingredients list and an associated tag from the initial recipe, and generate nutrition information and preparation instructions based on the ingredients list and the associated tag.

In a further embodiment, the recipe generation system is further configured to combine the nutrition information and the preparation instructions with the ingredients list and the associated tag to form a generated recipe.

In a still further embodiment the recipe generation system is further configured to store the generated recipe in the recipe selection database.

In another embodiment, the plurality of dietary requirements identify types of (i) recipes or (ii) food attributes that are associated with alleviating or resolving the symptom.

In yet another embodiment, the recipe requirement identifies one or more excluded ingredients, included ingredients, excluded ingredient types, included ingredient types, and/or nutritional requirements to comply with the dietary requirement.

In a further embodiment, a non-transitory, computer-readable medium storing instructions which, when executed by a processor, cause the processor to identify user information indicating a symptom affecting a user and identify a dietary requirement based on the symptom. The non-transitory, computer-readable medium may store further instructions which, when executed by the processor, cause the processor to identify a recipe requirement based on the dietary requirement and present a recipe recommendation to the user based on the recipe requirement.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 illustrates a system according to an exemplary embodiment of the present disclosure.

FIG. 2 illustrates database tables according to exemplary embodiments of the present disclosure.

FIG. 3 illustrates a method according to an exemplary embodiment of the present disclosure.

FIG. 4 illustrates a method according to an exemplary embodiment of the present disclosure.

### DETAILED DESCRIPTION OF EXAMPLE EMBODIMENTS

Patients diagnosed with certain conditions may suffer from symptoms that can negatively impact the patients' quality of life and may negatively impact treatment. Accordingly, intervention—in particular, nutritional intervention—that addresses these symptoms has been shown to: improve treatment response and adherence, reduce hospitalizations, enhance quality of life, and positively affect overall outcomes. However, prescribing such nutritional intervention based solely on a patient's diagnosis may not suffice to treat the patient's symptoms, because patients with similar diagnoses may experience different symptoms. Differences in symptoms between patients may relate to the specific treatment protocol a patient is undergoing and the unique pathophysiology of the patient. Additionally, even for an individual patient, the symptoms experienced may change over time, e.g., as the patient's treatment progress and/or the patient's condition or diagnosis changes. Therefore, any nutritional intervention should be personalized on a per-patient basis to address the specific symptoms that a patient is facing. One method of providing this level of personalization is to receive information from a patient regarding the symptoms that the patient is currently experiencing and to prepare nutritional intervention recommendations based on the patient's symptoms. In certain cases, further personalization may be provided based on additional information, such as the patient's diagnosis (e.g., cancer diagnosis), the patient's treatment protocol (e.g., chemotherapy, radiotherapy), the patient's medications, the patient's allergies, and the patient's food preferences. The nutritional intervention recommendations may be provided as recommended recipes that address the patient's symptoms.

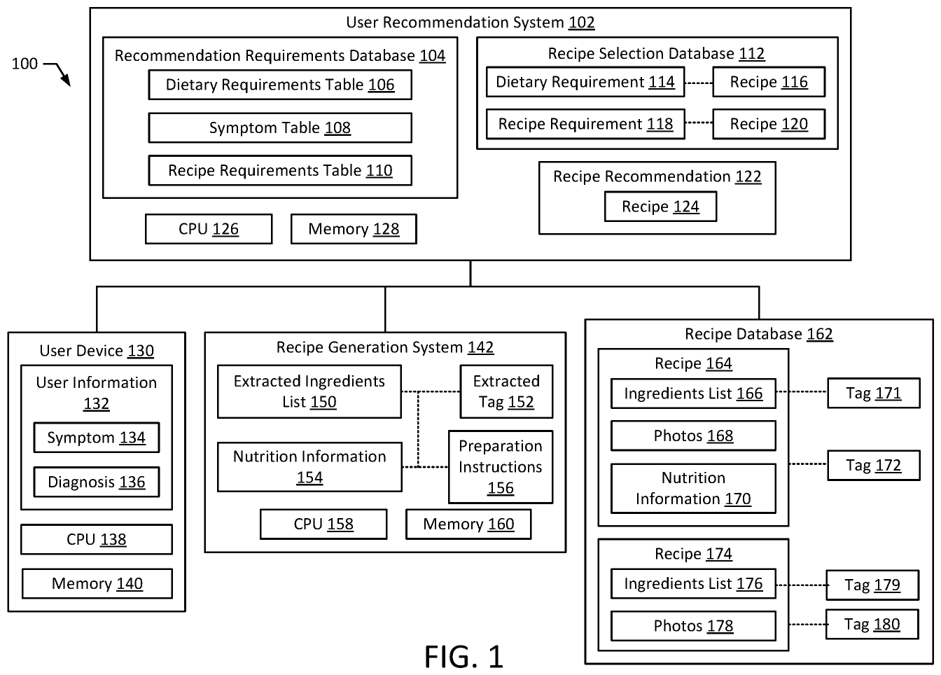

FIG. 1 illustrates a system 100 according to an exemplary embodiment of the present disclosure. The system 100 may be configured to identify recipes to alleviate symptoms experienced by a user (e.g., a patient undergoing treatment). The system 100 includes a user recommendation system 102, a user device 130, a recipe generation system 142, and a recipe database 162.

The user recommendation system 102 includes a recommendation requirements database 104, a recipe selection database 112, a recipe recommendation 122, a CPU 126, and a memory 128. The user recommendation system 102 may be configured to receive user information, such as the user information 132 from the user device 130, and generate a recipe recommendation 122 including at least one recipe 124 that complies with the user information 132. For example, the user information 132 may identify one or both of a symptom 134 and a diagnosis 136 (e.g., a medical diagnosis) of a user associated with the user device 130. The user device 130 may be implemented as a computing device, such as a computer, smartphone, tablet, smartwatch, or other wearable. The user device 130 may also be implemented as, e.g., a voice assistant configured to receive voice requests from a user and to process the requests either locally on a computer device proximate to the user or on a remote computing device (e.g., at a remote computing server).

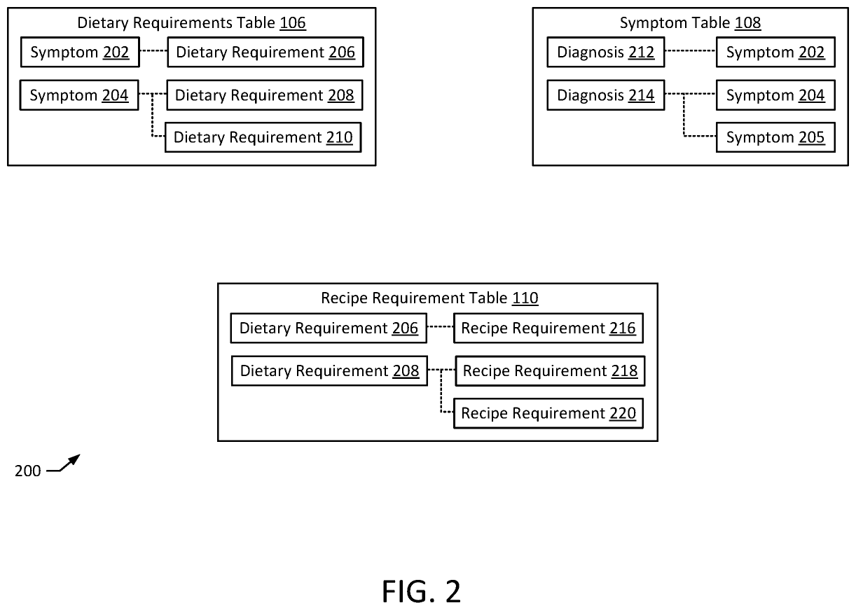

As explained further below, the recommendation requirements database 104 stores a dietary requirements table 106, a symptom table 108, and a recipe requirements table 110. In certain implementations, the symptom table 108 may be optional. For example, the recommendation requirements database 104 may, in certain implementations store a dietary requirements table 106 and a recipe requirements table 110 and may not store a symptom table 108. The dietary requirements table 106 may store a plurality of dietary requirements associated with certain symptoms. For example, as shown in the exemplary tables 200 of FIG. 2, the dietary requirements table 106 may store associations between symptoms 202, 204 and one or more dietary requirements 206, 208, 210. The dietary requirements 206, 208, 210 may identify requirements to help mitigate or resolve the associated symptoms 202, 204. Table 1 below depicts an exemplary dietary requirements table 106, with each of the numbered associated rules representing a separate dietary requirement 206, 208, 210 that may stored in association with a corresponding symptom 202, 204. Symptoms 3 and 4 represent template symptoms, which may be populated with symptom-based rules similar to the depicted poor appetite and dysphagia rules.

TABLE 1
Exemplary Dietary Requirements Table

| Symptom | Associated Rules |
|---|---|
| Poor Appetite | 1. Include High Protein Food |
| | 2. Include High Calorie Food |
| | 3. Exclude Low Fat Food |
| | 4. Exclude Low Calorie Food |
| Dysphagia | 1. Exclude Coarse Food |
| | 2. Exclude Hard Food |
| Symptom 3 | 1. Exclude Food Category Y |
| | 2. Include Food Category X |
| Symptom 4 | 1. Exclude Food Category X |
| | 2. Include Food Category Y |
| | 3. Exclude Food Category W |

The symptom table 108 may store a plurality of symptoms associated with certain diagnoses. For example, as shown in the exemplary tables 200 of FIG. 2, the symptom table 108 may store associations between diagnoses 212, 214 and one or more symptoms 202, 204, 205. The symptoms 202, 204, 205 may be common or uncommon associations with each diagnosis 212, 214, which may assist with generating a recipe recommendation 122 if a user only provides a diagnosis. In certain implementations, the symptom table 108 may be utilized to determine one or more symptoms 202, 204, 205 that a user may be experiencing regarding provided diagnosis information. For example, if a user only provides a diagnosis, the symptom table 108 may be utilized to predict symptoms 202, 204, 205 the user may be experiencing. In other instances where the user provides both a diagnosis and symptom information, the symptom table may be utilized to predict additional symptoms 202, 204, 205 the user may be experiencing. In still further examples, the recommendation requirements database 104 may not include a symptom table 108. In such examples, the user may be required to provide one or more symptoms 202, 204, 205 for further processing.

The recipe requirements table 110 may store information on certain recipe requirements. For example, as shown in the exemplary tables 200 of FIG. 2, the recipe requirement 110 may store dietary requirements 206 in association with one or more recipe requirements 216, 218, 220. The recipe requirements 216, 218, 220 may include specific foods to include or exclude according to the associated dietary requirements 206. The recipe requirements 216, 218, 220 may also include other limits or requirements for recipes to comply with the dietary requirements (e.g., calorie requirements, caffeine requirements, macronutrient requirements). Table 2 below depicts an exemplary recipe requirements table 110, with each numbered condition representing a potential recipe requirement 216, 218, 220 stored in association with a corresponding dietary requirement 206, 208. Similar to Table 1, the Food Category Y row may represent a template recipe requirement that may be populated with rule-based recipe requirements similar to the depicted hard food and low calorie food recipe requirements.

TABLE 2
Exemplary Recipe Requirement Table

| Rule | Recipe Requirements |
|---|---|
| Exclude Hard | Exclude (1) raw vegetables, (2) raw |
| Food | fruits, (3) nuts |
| Exclude Low | Exclude foods that provide < 400 |
| Calorie Foods | calories/serving |
| Exclude Food | Exclude (1) food type A, (2) food |
| Category Y | type B |

As explained further below, the user recommendation system 102 may utilize the information stored in the recommendation requirements database 104 to determine which types of recipes are acceptable or desirable for a user with identified symptoms 134, 202, 204 and/or diagnoses 136, 212, 214.

The recipe selection database 112 stores recipes 116, 120 in association with one or more dietary requirements 114, recipe requirements 118, and combinations thereof. For example, in a preferred embodiment, the recipe generation system 142 may add recipes 116, 120 to the recipe selection database 112 that comply with certain recipe requirements 118 and may store such associations in the recipe selection database 112. In additional or alternative embodiments, the user recommendation system 102 may identify recipes 116, 120 that comply with certain dietary requirements 114 and may store such associations in the recipe selection database 112. In certain such implementations, certain recipes 116, 120 may be stored in association with both a dietary requirement 114 and a recipe requirement 118.

The recipe database 162 stores recipes 164, 174 in association with one or more tags 171, 172, 179, 180. The recipes 164, 174 may include information on preparing one or more meals. Therefore, as depicted, the recipes 164, 174 include ingredients lists 166, 176 identifying the ingredients included in each meal. In certain implementations, the ingredients lists 166, 176 may be stored as tags (e.g., tags 171, 179) identifying one or more of the ingredients included in the recipes 164, 174. In still further implementations, the tags 171, 179 corresponding to the ingredients lists 166, 176 may provide additional information (e.g., ingredient categories, or food attributes associated with the ingredients of the ingredients lists 166, 176, as discussed further below in connection with the tags 172, 180. The recipes 164, 174 may store photos 168, 178 of the resulting meals, or of the meals in preparation. Certain recipes 164 may also include nutrition information 170 of the recipe. The tags 172, 180 may identify one or more categories or classifications applicable to each recipe 164, 174. For example, the tags 172, 180 may include one or more of Vegetarian, High Protein, Low Carb, Gluten Free, High Calorie, Low Calorie, Hard Food, and Soft Food, along with indications of recipe requirements 118 with which the recipes 164, 174 comply (e.g., certain food restrictions or inclusions).

The recipe generation system 142 may be configured to generate recipes based on information retrieved from the recipe database 162. For example, the recipe generation system 142 may extract limited information from the recipes 164, 174 and may generate or retrieve the remaining information to generate recipes for inclusion within the recipe selection database 112. In such examples, the recipe generation system 142 may receive a recipe (e.g., the recipe 164) from the recipe database 162 with an ingredients list 166 and one or more associated tags 171, 172. In particular, the recipe generation system 142 may extract limited information from the recipe database 162 according to tags 171, 172, 179, 180 associated with the recipes 164, 174. For example, when compiling recipes that comply with a recipe requirement 118 to include soft foods, the recipe generation system 142 may search the recipe database 162 for recipes with tags 171, 172, 179, 180 indicating soft food inclusion. Continuing this example, the recipe 164 may be for a banana smoothie and the tag 172 may therefore indicate that the recipe 164 includes soft foods. In implementations, where the ingredients lists 166, 176 are implemented as tags, the recipe generation system 142 may search the recipe database 162 for recipes 164, 174 with tags 171, 169 indicating ingredients that comply with the recipe requirement 118. The recipe generation system 142 may then extract information from the recipes with matching tags, such as the extracted ingredients list 150 and extracted tag 152. The recipe generation system 142 may then generate or retrieve nutrition information 154 and preparation instructions 156 for the recipe. The nutrition information 154 and the preparation instructions 156 may be generated based on the extracted ingredients list 150 and/or the extracted tag 152 without relying on further information from the recipe database 162. In preferred embodiments, the recipe generation system 142 may generate or retrieve nutrition information 154 and preparation instructions 156 for each generated recipe and may optionally generate or retrieve additional information regarding certain generated recipes, such as photos or a description of the recipe. After generation, the recipe generation system 142 may store the generated recipe as a recipe 116, 120 within the recipe selection database 112 in association with one or more recipe requirements 118 with which the generated recipe complies. Additionally or alternatively, the recipe generation system 142 may store the generated recipe as a recipe 116, 120 within the recipe selection database 112 in association with one or more dietary requirements 114 with which the generated recipe complies.

The user recommendation system 102, the user device 130, the recipe generation system 142, and the recipe database 162 may communicate via one or more networks, such as a local network and/or the internet. For example, the user recommendation system 102, the user device 130, the recipe generation system 142, and the recipe database 162 may communicate via one or more wired (e.g., Ethernet) or wireless (e.g., W-Fi, Bluetooth, cellular network) communication links.

One or more of the user recommendation system 102, the user device 130, the recipe generation system 142, and the recipe database 162 may be implemented by a computer system. For example, the CPUs 126, 138, 158 and the memories 128, 140, 160 may implement one or more features of the user recommendation system 102, the user device 130, and the recipe generation system 142. For example, the memories 128, 140, 160 may contain instructions which, when executed by the CPUs 126, 138, 158, cause the CPUs 126, 138, 158 to perform one or more of the operational features of the user recommendation system 102, the user device 130, and/or the recipe generation system 142. Similarly, although not depicted, one or more functions of the recipe database 162 may be implemented by a CPU and/or a memory.

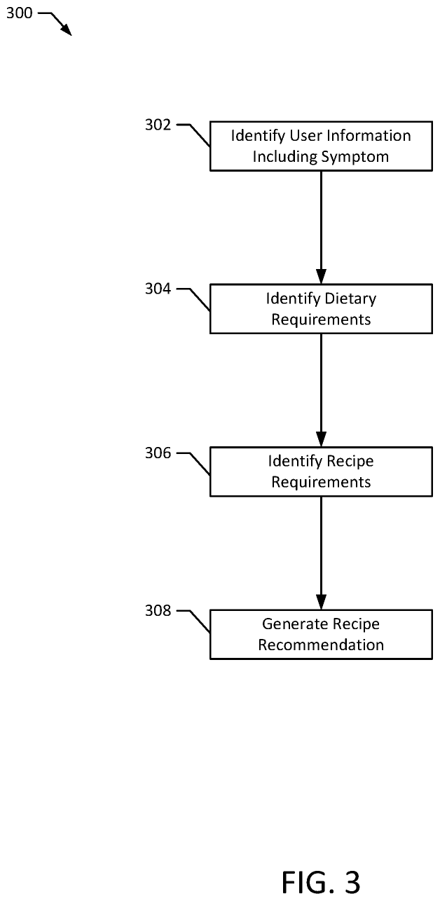

FIG. 3 illustrates a method 300 according to an exemplary embodiment of the present disclosure. The method 300 may be performed to receive and process user information 132 from a user device 130 to generate the recipe recommendation 122. For example, the method 300 may be performed by the user recommendation system 102 to generate the recipe recommendation 122. The method 300 may be implemented on a computer system, such as the system 100. For example, the method 300 may be implemented by the user recommendation system 102, the user device 130, the recipe generation system 142, and/or the recipe database 162. The method 300 may also be implemented by a set of instructions stored on a computer readable medium that, when executed by a processor, cause the computer system to perform the method. For example, all or part of the method 300 may be implemented by the CPUs 126, 138, 158 and the memories 128, 140, 160. Although the examples below are described with reference to the flowchart illustrated in FIG. 3, many other methods of performing the acts associated with FIG. 3 may be used. For example, the order of some of the blocks may be changed, certain blocks may be combined with other blocks, one or more the blocks may be repeated, and some of the blocks described may be optional.

The method 300 begins at the user recommendation system 102 receiving user information 132 indicating a symptom 134 (block 302). For example, the user recommendation system 102 may receive the user information 132 from a user device 130. The symptom 134 may identify one or more symptoms that a user associated with the user device 130 is currently experiencing. For example, the user may be diagnosed with a particular disease or medical condition and the symptom may result from that medical condition and/or from treatment associated with the medical condition. In particular, a user may be diagnosed with lower gastrointestinal cancer and may be suffering from constipation as a result of this diagnosis. The user may provide the symptom 134 in order to receive recipe recommendations 122 containing recipes 124 that will help alleviate or remove the symptom 134. In other implementations, the user information 132 may be received from a medical professional, such as a medical professional treating the user. Although depicted in the singular, the user information 132 may include more than one symptom 134. Additionally, in other implementations, the user information 132 may include a diagnosis 136 specifying the disease or medical condition applicable to the associated user for which the recipe recommendation 122 is being generated, which may, in certain implementations, be used to identify symptoms 202, 204, 205, as discussed above.

The user recommendation system 102 may then identify dietary requirements 114, 206, 208, 210 associated with the user information 132 (block 304). For example, the user recommendation system 102 may query a dietary requirements table 106 with the provided symptom 134 from the user information 132 for one or more dietary requirements 206, 208, 210 associated with the provided symptom 134. Continuing the above example, based on a received symptom 134 indicating constipation, the dietary requirements table 106 may include a dietary requirement 206, 208, 210 that the user include high fiber food in their diet. In implementations where the user information 132 includes only a diagnosis 136, the user recommendation system 102 may identify one or more symptoms 202, 204, 205 associated with the diagnosis 136 in the symptom table 108. For example, if the user in the previous example provided a diagnosis 136 to the user recommendation system 102 indicating lower gastrointestinal cancer, but did not identify a symptom 134 for which the recipe recommendation 122 is to be generated, the user recommendation system 102 may identify constipation as a probable symptom 202, 204, 205 associated with such a diagnosis 136. Based on this probable symptom 202, 204, 205, the user recommendation system 102 may generate the dietary requirements 206, 208, 210 as described above.

The user recommendation system 102 may then generate recipe requirements 216, 218, 220, 118 (block 306). As explained above, the recipe requirements 216, 218, 220, 118 may identify one or more food-based or other restrictions for recipes 164, 174 stored in the recipe database 162 and/or the recipe selection database 112 to follow the previously-generated dietary requirements 114, 206, 208, 210. To generate the recipe requirements 216, 218, 220, 118, the user recommendation system 102 may consult the recipe requirement table 110 of the recommendation requirements database 104. For example, the user recommendation system 102 may identify one or more recipe requirements 216, 218, 220 corresponding to the previously-generated dietary requirements 206, 208, 210 within the recipe requirement table 110. Continuing the previous examples, based on the dietary requirement 206, 208, 210 that the user include high fiber food, the user recommendation system 102 may identify a recipe requirement 216, 218, 220 requiring a fiber level 5g per serving.

In certain implementations, one or more of blocks 302, 304, and 306 may be optional. For example, if the user recommendation system 102 has already received user information 132 from a user, the method 300 may begin with identifying dietary requirements at block 304 based on the previously-received user information 132. Similarly, if the user recommendation system 102 stores dietary requirements 114, 206, 208, 210 and/or recipe requirements 216, 218, 220, 118 that were previously generated for a user, the user recommendation system 102 may utilize the previously-stored requirements at block 308 rather than regenerating them at blocks 304, 306. However, in certain implementations (e.g., when a user updates their user information 132 if their symptoms change), the user recommendation system 102 may receive user information 132 corresponding to a user for which user information 132 was previously received. In such implementations, the user recommendation system 102 may proceed to execute blocks 302, 304, 306 to update the dietary requirements 114, 206, 208, 210 and/or recipe requirements 216, 218, 220, 118 based on the updated user information 132.

The user recommendation system 102 may then generate the recipe recommendation 122 (block 308). The recipe recommendation 122 may be generated to include one or more recipes 124 that comply with the recipe requirements 216, 218, 220. In particular, the user recommendation system 102 may identify one or more recipes 116, 120 within the recipe selection database 112 that have an associated recipe requirement 118 similar or identical to the generated recipe requirements 216, 218, 220. The recipe recommendation 122 may additionally or alternatively be generated by identifying recipes 164, 174 within the recipe database 162 that comply with the recipe requirements 216, 218, 220. For example, such recipes 164, 174 may be identified based on one or more of the ingredients lists 166, 176, nutrition information 170, and/or the tags 172, 180. In certain implementations, the recipe generation system 142 may be configured to further generate the recipe(s) 124 for inclusion within the recipe recommendation 120 based on the identified recipes 164, 174 of the recipe database 160.

In other implementations, the user recommendation system 102 may not generate the recipe requirements 216, 218, 220 at block 306 and may instead identify a recipe 116 within the recipe selection database 112 with a similar or identical dietary requirement 114 to the dietary requirement 206, 208, 210 identified in block 304.

Once generated, the recipe recommendation 122 may be presented to the user (e.g., via the user device 130). One or more recipes 124 may be included within the recipe recommendation 120 to present to the user via a user interface, which the user may use to view photos and other information regarding the recipes 124 (e.g., the extracted ingredient list 150 and/or the generated nutrition information 154 and preparation instructions 156). In certain implementations, where the user device 130 and/or the user information 132 has food preference information corresponding to the user, the recipes 124 are included within the recipe recommendation 122 and/or the recipes 124 displayed to the user via the user device 130 may be filtered to account for the provided food preference information (e.g., by removing recipes that contain ingredients identified as disliked by the user). In certain implementations, food preference information may be included in the user information 132 and may be included as part of the recipe requirements 216, 218, 220 identified at block 306. Further, the recipe recommendation 122 may be generated as part of a meal plan generated for the user. For example, the meal plan may be generated to include recipes for a week of food consumption for a period of time for the user (e.g., breakfast, lunch, and dinner for 7 days) according to the user's dietary needs and/or preferences.

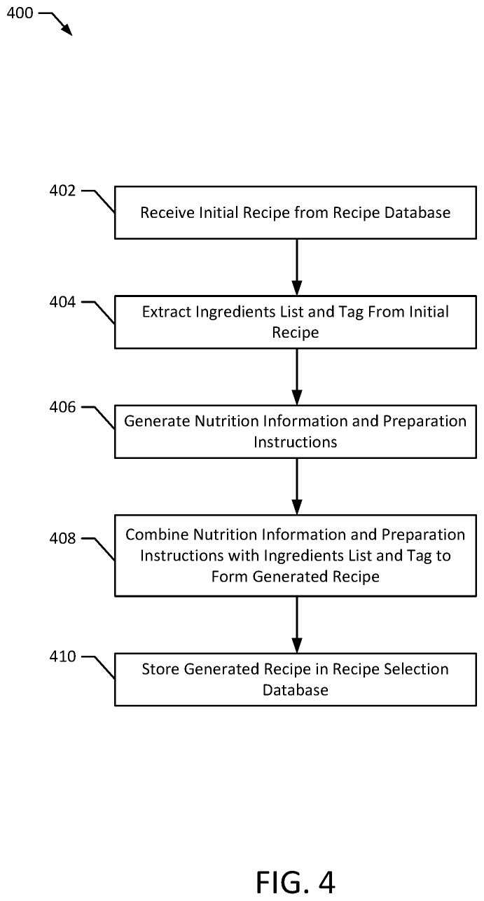

FIG. 4 illustrates a method 400 according to an exemplary embodiment of the present disclosure. The method 400 may be performed by the user recommendation system 102, the recipe generation system 142, and the recipe database 162 to add recipes 116, 120 to the recipe selection database 112. In certain implementations, the steps of the method 400 may be performed prior to execution of the method 300. For example, the method 400 may be performed to generate the recipes 116, 120 and associated recipe requirements 118 and/or dietary requirements 114 of the recipe selection database 112 for subsequent use in executing the method 300. The method 400 may be implemented on a computer system, such as the system 100. For example, the method 400 may be implemented by the user recommendation system 102, the user device 130, the recipe generation system 142, and/or the recipe database 162. The method 400 may also be implemented by a set of instructions stored on a computer readable medium that, when executed by a processor, cause the computer system to perform the method. For example, all or part of the method 400 may be implemented by the CPUs 126, 138, 158 and the memories 128, 140, 160. Although the examples below are described with reference to the flowchart illustrated in FIG. 4, many other methods of performing the acts associated with FIG. 4 may be used. For example, the order of some of the blocks may be changed, certain blocks may be combined with other blocks, one or more the blocks may be repeated, and some of the blocks described may be optional.

The method 400 begins with the recipe generation system 142 receiving an initial recipe from the recipe database 162 (block 402). The recipe generation system 142 may receive the initial recipe as a potential basis for a generated recipe for inclusion within the recipe selection database 112. In certain implementations, the recipe generation system 142 may receive recipes from the recipe database 162 at regular intervals (e.g., every day, week, month, quarter). In other implementations, the recipe generation system 142 may receive the initial recipe when a new recipe 164, 174 is added to the recipe database 162. In certain implementations, the recipe generation system 142 may request the initial recipe from the recipe database 162 (e.g., by identifying one or more tags 171, 172, 179, 180 for which recipes are desired). In certain implementations, the recipe generation system 142 may receive the initial recipe over a network connection (e.g., an internet connection or a local area network connection) with the recipe database. In such implementations, the initial recipe may be received according to an application programming interface (API). The initial recipe may be implemented similar to the recipes 164, 174 and may accordingly include one or more of an ingredients list 166, 176, photos 168, 178, and nutrition information 170.

The recipe generation system 142 may then extract an extracted ingredients list 150 and an extracted tag 152 from the initial recipe (block 404). The recipe generation system 142 may copy this information from the recipe itself (e.g., from the ingredients list 166, 176 and tags 171, 172, 179, 180 stored in association with the initial recipe in the recipe database 162. For example, where the recipe 164 is the initial recipe, the extracted ingredients list 150 may include the same ingredients as the ingredients list 166 and extracted tag 152 may include one or both of the tags 171, 172.

Based on the extracted ingredients list 150 and the extracted tag 152, the recipe generation system 142 may generate nutrition information 154 and preparation instructions 156 (block 406). In certain implementations, the nutrition information 154 and the preparation instructions 156 may be retrieved from a recipe generation service. In other implementations, the recipe generation system 142 may generate nutrition information 150 based on the ingredients included within the extracted ingredients list 150 (e.g., based on the caloric and other nutrition information for the constituent ingredients and amount information for each ingredient contained within the extracted ingredient list 150). The recipe generation system 142 may also generate preparation instructions 156 based on prior processed recipes and/or one or more programmatic heuristics.

The recipe generation system 142 may then combine the nutrition information 154 and the preparation instructions 156 with the extracted ingredients list 150 and the extracted tag 152 to form a generated recipe (block 408). The generated recipe may include a data structure similar to that of the recipe 164. For example, the extracted ingredients list 150, the nutrition information 154, and the preparation instructions 156 may be stored within the generated recipe and the extracted tag 152 may be stored in association with the generated recipe. In certain implementations, additional information may be generated for inclusion within the generated recipe, such as photos, as discussed above.

The generated recipe may then be stored in the recipe selection database 112 (block 410). For example, after generating the generated recipe, the recipe generation system 142 may transmit the generated recipe to the user recommendation system 102 for storage in the recipe selection database 112. Once stored, the generated recipe may be utilized in subsequent recipe recommendation 122 generation procedures. In particular, the generated recipe may then be analyzed or otherwise utilized during performance of the method 300 (e.g., as a recipe 116, 120 in the recipe selection database 112).

The recipe selection database 112 may store the generated recipe in association with the extracted tag 152. For example, in certain implementations, one or both of the recipe requirement 118 and the dietary requirement 114 may correspond to an extracted tag 152 of the generated recipe. By storing generated recipes for future use in the recipe selection database 112, the user recommendation system 102 may be able to generate recipe recommendations 122 without having to rely on the recipe generation system 142 and/or the recipe database 162. Accordingly, such an implementation may reduce the complexity required to generate a recipe recommendation 122, which may increase responsiveness and reduce the time required to generate recipe recommendations 122. In certain implementations, the recipe selection database 112 may also store the recipes 116, 120 in association with tags that enable artificial intelligence-based improvements to the recipes 124 included in the recipe recommendation 122 (e.g., popularity or user ratings of the recipes 116, 120).

In certain implementations, more than one initial recipe may be received by the recipe generation 142 at block 402. In such implementations, the recipe generation system 142 may repeat processing at blocks 404, 406, and 408 to process each received initial recipe in order to generate a generated recipe corresponding to each of the received initial recipes. At block 410, the generated recipes may then be stored in the recipe selection database 112.

In further implementations, the method 400 may be performed prior to receiving user information 132. For example, the method 400 may be performed initially to populate the recipe selection database 112 with recipes 116, 120 for each of the recipe requirements 216, 218, 220 within the recipe requirement table 110, or a subset thereof, and/or for each dietary requirements 206, 208, 210 within the dietary requirements table 106, or a subset thereof.

In still further implementations, blocks 406 and 408 may be optional. For example, the recipe database 162 may instead transmit recipes 164, 174 that comply with designated recipe requirements 216, 218, 220 to the user recommendation system 102 for inclusion within the recipe recommendation 122. In such implementations, the system 100 may not include a recipe generation system 142 and may, in additional or alternative implementations, also lack the recipe selection database 112.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A method comprising:
identifying user information indicating a symptom affecting a user;
identifying a dietary requirement based on the symptom;
identifying a recipe requirement based on the dietary requirement; and
presenting a recipe recommendation to the user based on the recipe requirement.

2. The method of claim 1, wherein identifying the user information further comprises one or both of:
receiving user information from the user indicating the symptom; and
identifying previously-received user information indicating the symptom.

3. The method of claim 1, further comprising:
identifying a plurality of recipes within a recipe selection database that comply with the recipe requirement;
selecting at least one selected recipe from among the plurality of recipes; and
including the at least one selected recipe in the recipe recommendation.

4. The method of claim 3, wherein the at least one selected recipe is selected from among the plurality of recipes according to a user preference associated with the user information.

5. The method of claim 1, further comprising:
receiving an initial recipe from a recipe database;
extracting an ingredients list and an associated tag from the initial recipe; and
generating nutrition information and preparation instructions based on the ingredients list and the associated tag.

6. The method of claim 5, further comprising:
combining the nutrition information and the preparation instructions with the ingredients list and the associated tag to form a generated recipe.

7. The method of claim 6, further comprising:
storing the generated recipe in a recipe selection database.

8. The method of claim 1, wherein the dietary requirement identifies types of (i) recipes or (ii) food attributes that are associated with alleviating or resolving the symptom.

9. The method of claim 1, wherein the recipe requirement identifies one or more excluded ingredients, included ingredients, excluded ingredient types, included ingredient types, and/or nutritional requirements to comply with the dietary requirement.

10. The method of claim 1, wherein the symptom includes at least one condition selected from the group consisting of poor appetite, xerostomia, weight loss, mucositis, nausea, dysphagia, constipation, and diarrhea.

11. A system comprising:
a processor; and
a memory storing instructions which, when executed by the processor, cause the processor to implement:
a recommendation requirements database including at least (i) a dietary requirements table storing a plurality of dietary requirements associated with one or more symptoms and (ii) a recipe requirements table storing a plurality of recipe requirements associated with the dietary requirements; and
a user recommendation system configured to:
identify user information indicating a symptom affecting a user;
identify, within the dietary requirements table, a dietary requirement based on the symptom;
identify, within the recipe requirements table, a recipe requirement based on the dietary requirement; and
presenting a recipe recommendation to the user based on the recipe requirement.

12. The system of claim 11, wherein the user recommendation system is configured, to identify the user information by:
receiving user information from the user indicating the symptom; and
identifying previously-received user information indicating the symptom.

13. The system of claim 11, wherein the memory stores further instructions which, when executed by the processor, cause the processor to further implement:
a recipe selection database storing a plurality of recipes associated with the plurality of recipe requirements, and
wherein the user recommendation system is further configured to:
identify a plurality of recipes within the recipe selection database that comply with the recipe requirement;
select at least one selected recipe from among the plurality of recipes; and
include the at least one selected recipe in the recipe recommendation.

14. The system of claim 13, wherein the at least one selected recipe is selected from among the plurality of recipes according to a user preference associated with the user information.

15. The system of claim 13, wherein the memory stores further instructions which, when executed by the processor, cause the processor to further implement:
a recipe generation system configured to:
receive an initial recipe from a recipe database;
extract an ingredients list and an associated tag from the initial recipe; and
generate nutrition information and preparation instructions based on the ingredients list and the associated tag.

16. The system of claim 15, wherein the recipe generation system is further configured to:
combine the nutrition information and the preparation instructions with the ingredients list and the associated tag to form a generated recipe.

17. The system of claim 16, wherein the recipe generation system is further configured to:
store the generated recipe in the recipe selection database.

18. The system of claim 11, wherein the plurality of dietary requirements identify types of (i) recipes or (ii) food attributes that are associated with alleviating or resolving the symptom.

19. The system of claim 11, wherein the recipe requirement identifies one or more excluded ingredients, included ingredients, excluded ingredient types, included ingredient types, and/or nutritional requirements to comply with the dietary requirement.

20. A non-transitory, computer-readable medium storing instructions which, when executed by a processor, cause the processor to:
identify user information indicating a symptom affecting a user;
identify a dietary requirement based on the symptom;
identify a recipe requirement based on the dietary requirement; and
present a recipe recommendation to the user based on the recipe requirement.
